(19) United States
(12) Patent Application Publication
(10) Pub. No.: US 2009/0280189 A1
(43) Pub. Date: Nov. 12, 2009

(54) System for the liberation of an active principle and its use

(75) Inventors: Klaus-Dieter Kühn, Marburg-Elnhausen (DE); Sebastian Vogt, Erfurt (DE)

Correspondence Address: NORRIS, MCLAUGHLIN & MARCUS, PA, 875 THIRD AVENUE, 18TH FLOOR, NEW YORK, NY, 10022, US

(73) Assignee: HERAEUS KULZER GMBH, Hanau (DE)

(21) Appl. No.: 12/506,388

(22) Filed: Jul. 21, 2009

Related U.S. Application Data
(62) Division of application No. 11/509,252, filed on Aug. 24, 2006.

(30) Foreign Application Priority Data
Aug. 25, 2005 (DE) 10 2005 040 429.4

(51) Int. Cl. A61K 33/24; A61K 31/7036

(52) U.S. Cl. 424/617; 514/40

(57) ABSTRACT

A local system for the liberation of an active principle is described which consists of spherical bodies which are composed of polymethyl methacrylate or polymethyl methacrylate co-methyl acrylate and, if necessary, zirconium dioxide and/or barium sulphate and a pharmaceutical active principle, which contains at least one hemostyptically effective compound stable at least up to 120° C., preferably a calcium salt. The local system for the liberation of an active principle is provided as medical product or drug.

This application is a division of U.S. patent application Ser. No. 11/509,252, filed on Aug. 24, 2006, now pending, which, in turn, claims priority of German Patent Application No. DE 10 2005 040 429.4, filed on Aug. 25, 2005.

The subject matter of the invention is a locally effective system for the liberation of an active principle which consists of bodies which are composed essentially of polymethyl methacrylate or polymethyl methacrylate co-methyl acrylate and zirconium dioxide or barium sulphate and a pharmaceutical active principle.

Even today, the treatment of osteomyelitis provides one of the most difficult challenges in bone surgery. Osteomyelitis can have hematogenic, posttraumatic or postoperative causes. The chronic form of osteomyelitis is particularly difficult to treat and can in extreme cases lead to the loss of limbs and even to sepsis.

Commonly, surgical remediation by radical debridement is effected. During this process, the infected and/or necrotic bone is largely excised. Subsequently, the bone cavity is filled with a local antibiotic carrier or treated by repeated suction/irrigation drainage. Through the local liberation of large quantities of antibiotics, the bacterial germs remaining also in the adjacent bone areas are effectively controlled by using a sufficiently bone penetrative bactericidal antibiotic such as gentamicin sulphate and clintamycin hydrochloride.

Spherical local systems for the liberation of an active principle composed of polymethyl methacrylate, zirconium dioxide and an antibiotic were first described by Klaus Klemm in 1975 (DE 23 20 373). This concept proved basically successful but had the disadvantage that only a small part of the active principle contained in the spheres was liberated.

As a further development of this active principle carrier, Heuser and Dingeldein suggested in 1978 to add glycine or other amino acids to improve the liberation of the antibiotic (DE 26 51 441). Following contact with discharge from the wound, the incorporated amino acids dissolve and form pore systems from which the active principle is able to diffuse out. As a result, an improved liberation of the active principle was achieved.

Local systems for the liberation of an active principle which are composed mainly of polymethyl methacrylate, an x-ray opaquer and an antibiotic can be produced either by a special injection moulding process (DE 23 20 373) or by casting antibiotic-containing polymethyl methacrylate bone cement in special molds (EP 0 796 712).

At present a local system for the liberation of an active principle consisting of spheres which are composed of polymethyl methacrylate, zirconium dioxide, glycine and gentamicin which are joined to each other by a polyfilic surgical steel wire is being made by Heraeus Kulzer GmbH and marketed by Biomet under the name SeptopalO.

Users of this local system for the liberation of an active principle have variously reported the observation that the local antibiotic therapy is particularly successful if a hematoma is formed around the active principle carrier immediately after the application of the active principle carrier. This observation can be explained by the fact that coagulated blood delays the diffusion of gentamicin and thus hinders the removal of the active principle. As a result, the gentamicin remains in the previous surgically remediated bone cavity for a longer period and thus controls the residual bacterial germs for a long duration.

The invention is based on the task of developing a system for the liberation of an active principle which, on the one hand, exhibits a retarded liberation of active principle and, on the other hand, promotes the coagulation of the blood in the immediate vicinity of the active principle carriers.

The task has been achieved by developing a (local) system for the liberation of an active principle consisting of bodies which are composed essentially of polymethyl methacrylate or polymethyl methacrylate co-methyl acrylate, zirconium dioxide or barium sulphate and a pharmaceutical active principle, but which are characterised in that at least one hemostyptically effective compound stable up to 120° C. is contained therein. As a result of the hemostyptically effective compound, the formation of hematoma is encouraged. It is essential for the invention that this compound is stable up to at least 120° C. to allow the manufacture of the active principle carrier by injection molding. The bodies may preferably be spherical.

Inorganic or organic calcium salts are preferred as hemostyptically effective compounds. It is a fact known as such that dissolved calcium ions are able to accelerate the coagulation of the blood. Calcium ions are an essential component at several points of the coagulation cascade. They contribute to the activation of factor VII and factor IX and thus during the formation of the prothrombin activator. Calcium ions are, moreover, essential in the action of thrombin onto fibrinogen to form fibrin monomers which in turn form the fibrin network with the contribution of the active factor XIII.

The at least one hemostyptically effective compound is preferably contained in a quantity of 0.1-60.0 percent by mass, based on the spherical bodies.

Where calcium salts are involved, these should have a solubility in water at room temperature of at least 0.5 g per liter.

The calcium salts calcium sulphate, calcium sulphate dihydrate, calcium sulphate hemihydrate, calcium hydroxide, calcium dihydrogen phosphate, calcium lactate, calcium gluconate and calcium acetate are particularly preferred. In addition, other pharmaceutically acceptable calcium salts can be used. Thus, it is equally possible to use also calcium salts of amino acids, aldonic acids and uronic acids.

The calcium salt concerned can be microporous. Microporous calcium sulphate dihydrate is particularly preferred, especially microporous calcium sulphate dihydrate, in the microporous cavity system of which a pharmaceutical active principle from the group of antibiotics, antiphlogistics, hormones and carcinostatics is contained. These active principles can be introduced into the calcium dihydrate e.g. by impregnation. It is also possible to precipitate active principle salts with a low solubility in water directly into the microporous calcium sulphate dihydrate.

The calcium salt can completely replace zirconium dioxide or barium sulphate. The system for the liberation of an active principle is then composed merely of polymethyl methacrylate or polymethyl methacylate co-methyl acrylate, the calcium salt and the active principle. The calcium salt basically satisfies also the function of an x-ray opaquer. However, the absorption of the x-rays is noticeably less marked than in the case of zirconium dioxide or barium sulphate. The system for the liberation of an active principle is held together by the polymethyl methacrylate or polymethyl methacrylate co-methyl acrylate.

The application usually takes place in such a way that the local system for the liberation of an active principle is produced or provided as a medical product or drug.

The invention will be explained by the following examples without, however, limiting the invention.

### EXAMPLE 1

A mixture of 854.0 g polymethyl methacrylate co-methyl acrylate (molecular weight approx. 900,000 g/mole), 89.0 g zirconium dioxide, 42.0 g gentamicin sulphate (activity coefficient 600), 10.0 glycine and 5.0 g calcium sulphate dihydrate is made by intense grinding. From this mixture, approximately spherical bodies with a diameter of 7 mm are sprayed by means of an injection molding device onto a polyfilic surgical steel wire. These bodies have a mass of 240 mg.

### EXAMPLE 2

A mixture of 854.0 g polymethyl methacrylate co-methyl acrylate (molecular weight approx. 900,000 g/mole), 89.0 g zirconium dioxide, 42.0 g gentamicin sulphate (activity coefficient 600), 5.0 glycine and 10.0 g calcium sulphate dihydrate is made by intense grinding. From this mixture, approximately spherical bodies with a diameter of 7 mm are sprayed by means of an injection molding device onto a polyfilic surgical steel wire. These bodies have a mass of 240 mg.

### EXAMPLE 3

A mixture of 769.0.0 g polymethyl methacrylate co-methyl acrylate (molecular weight approx. 900,000 g/mole), 89.0 g zirconium dioxide, 42.0 g gentamicin sulphate (activity coefficient 600) and 100.0 g calcium sulphate dihydrate is made by intense grinding. From this mixture, approximately spherical bodies with a diameter of 7 mm are sprayed by means of an injection molding device onto a polyfilic surgical steel wire. These bodies have a mass of 240 mg.

### EXAMPLE 4

A mixture of 854.0 g polymethyl methacrylate co-methyl acrylate (molecular weight approx. 900,000 g/mole), 42.0 g gentamicin sulphate (activity coefficient 600) and 104.0 g calcium sulphate dihydrate is made by intense grinding. From this mixture, approximately spherical bodies with a diameter of 7 mm are sprayed by means of an injection molding device onto a polyfilic surgical steel wire. These bodies have a mass of 240 mg.

1 A method of treating a disorder in a patient with a pharmaceutical active principle effective to treat said disorder, said method comprising administering to said patient an effective amount therefor of a composition of matter in the form of bodies comprising polymethyl methacrylate or polymethyl methacrylate co-methylacrylate, a pharmaceutical active principle, and at least one hemostypticaly effective compound stable up to at least 120° C., and, optionally zirconium dioxide and/or barium sulphate.

2. The method according to claim 1, wherein the composition of matter comprises 0.1-60.0 percent by weight of the at least one hemostyptically effective compound.

3. The method according to claim 1, wherein the at least one hemostyptically effective compound comprises at least one inorganic or organic calcium salt.

4. The method according to claim 3, wherein the calcium salt has a solubility in water at room temperature of at least 0.5 g per liter.

5. The method according to claim 3, wherein the calcium salt is selected from the group consisting of calcium sulphate, calcium sulphate dihydrate, calcium sulphate hemihydrate, calcium dihydrogen phosphate, calcium lactate, calcium gluconate and calcium acetate.

6. The method according to claim 3, wherein the calcium salt concerned is microporous.

7 The method according to claim 6, wherein the calcium salt is microporous calcium sulphate dehydrate and the pharmaceutical active principle is selected from the group consisting of antibiotics, antiphlogistics, hormones and carcinostatics.

8. The method according to claim 1, wherein the composition of matter does not comprise the zirconium dioxide or barium sulphate.

9. The method according to claim 1, wherein the composition of matter is in the form of spherical bodies.

10. A method according to claim 1, wherein the composition of matter is a pharmaceutical composition.

11. The method according to claim 1, wherein the disorder is osteomyelitis, and the pharmaceutical active principle is an antibiotic.

12. The method according to claim 11, which comprises administering the composition of matter to said patient by introducing the composition of matter into a bone cavity produced in said patient.
